(19) United States
(12) Patent Application Publication
(10) Pub. No.: US 2024/0082275 A1
(43) Pub. Date: Mar. 14, 2024

(54) Pharmaceutical formulations comprising 5-Chloro-N4-[2-(dimethylphosphoryl)phenyl]-N2-{2-methoxy-4-[4-(4-methylpiperazin-1-yl)piperidin-1-yl]phenyl}pyrimidine-2,4-diamine

(71) Applicant: Takeda Pharmaceutical Company Limited, Osaka (JP)

(72) Inventors: Dauntel S. Verwijs, Sudbury, MA (US); Samir Desai, Suwanee, GA (US); Pradeep K. Sharma, Westford, MA (US); Leonard W. Rozamus, Andover, MA (US); Jeff Williamson, Palm Harbor, FL (US); Danica Cartwright, Land O Lakes, FL (US); Parag Ved, Lutz, FL (US)

(21) Appl. No.: 18/508,499

(22) Filed: Nov. 14, 2023

Related U.S. Application Data
(63) Continuation of application No. 17/726,736, filed on Apr. 22, 2022, now abandoned.
Application No. 17/726,736 is a continuation of application No. 17/474,246, filed on Sep. 14, 2021, now abandoned.
Application No. 17/474,246 is a continuation of application No. 17/193,502, filed on Mar. 5, 2021, now abandoned.
Application No. 17/193,502 is a continuation of application No. 15/914,899, filed on Mar. 7, 2018, now abandoned.
(60) Provisional application No. 62/569,954, filed on Oct. 9, 2017.
(60) Provisional application No. 62/491,179, filed on Apr. 27, 2017.
(60) Provisional application No. 62/468,696, filed on Mar. 8, 2017.

(51) Int. Cl. A61K 31/675; A61K 9/20; A61K 9/28; A61K 31/662; A61P 35/00

(52) U.S. Cl. CPC A61K 31/675; A61K 9/2009; A61K 9/2013; A61K 9/2018; A61K 9/2036; A61K 9/2054; A61K 9/2059; A61K 9/2095; A61K 9/284; A61K 31/662; A61P 35/00

(57) ABSTRACT

This invention relates to a pharmaceutical composition comprising 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine as the active pharmaceutical ingredient, and therapeutic uses of the pharmaceutical formulation. In particular, the invention is directed to tablets comprising the pharmaceutical composition, methods of preparing the tablets, and therapeutic uses thereof.

This application claims priority to U.S. Provisional Application Nos. 62/468,696, filed Mar. 8, 2017, 62/491,179, filed Apr. 27, 2017, and 62/569,954, filed Oct. 9, 2017, the entireties of which are incorporated herein by reference.

### FIELD OF THE INVENTION

This invention relates to a pharmaceutical composition comprising 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (also referred to as “AP26113” and “brigatinib”) as the active pharmaceutical ingredient. In particular, the invention is directed to tablets comprising the pharmaceutical composition and to methods of preparing the tablets. The invention further relates to therapeutic uses of the pharmaceutical formulation.

### BACKGROUND TO THE INVENTION

Brigatinib has the chemical formula C₂₉H₃₉CIN₇O₂P, which corresponds to a formula weight of 584.09 g/mol. Its chemical structure is shown below.

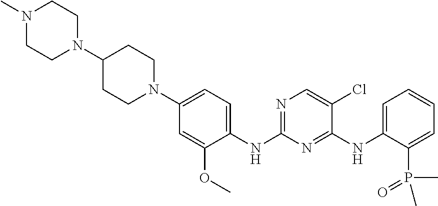

Brigatinib is a multi-targeted tyrosine-kinase inhibitor useful for the treatment of non-small cell lung cancer (NSCLC) and other diseases. It is a potent inhibitor of ALK (anaplastic lymphoma kinase) and is in clinical development for the treatment of adult patients with ALK-driven NSCLC. Crizotinib (XALKORI®) is an FDA approved drug for first-line treatment of ALK-positive NSCLC, but as stated in Shaw et al., New Eng. J. Med. 370:1 189-97 2014 “Despite initial responses to crizotinib, the majority of patients have a relapse within 12 months, owing to the development of resistance.” Brigatinib is thus a new and effective therapy for cancer patients with ALK-positive cancers.

Brigatinib is also potentially useful for treating other diseases or conditions in which ALK or other protein kinases inhibited by brigatinib are implicated. Such kinases and their associated disorders or conditions are disclosed in WO 2009/143389.

Brigatinib is disclosed in WO 2009/143389, which is incorporated herein by reference. Example 122 of WO 2009/143389 describes the synthesis of brigatinib and indicates that the product is obtained as an off-white solid. Several polymorphic forms of Brigatinib are described in WO 2016/065028, which is incorporated herein by reference.

In order that the therapeutic benefits of brigatinib may be delivered to patients in need thereof, there is a need to formulate brigatinib into pharmaceutical compositions, particularly solid dosage forms suitable for oral administration. Among the difficulties in identifying optimised pharmaceutical compositions comprising brigatinib are the need to ensure the chemical and physical stability of the active ingredient and excipients, the homogeneity of the blended pharmaceutical composition, the hardness and strength of the solid dosage forms, together with effective dissolution and bioavailability properties.

### SUMMARY OF THE INVENTION

The invention provides a pharmaceutical composition comprising:
(i) about 10 to about 40 wt % of 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib);
(ii) about 20 to about 50 wt % of lactose monohydrate; and
(iii) about 15 to about 50 wt % of microcrystalline cellulose.

The invention further provides a pharmaceutical composition comprising:
(i) about 10 to about 40 wt % 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib); and
(ii) about 0.2 to about 5 wt % hydrophobic colloidal silica.

The invention further provides a pharmaceutical composition comprising:
(i) about 10 to about 40 wt % 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib); and
(ii) about 0.5 to about 5 wt % of sodium starch glycolate.

The invention provides a pharmaceutical composition comprising:
(i) about 10 to about 40 wt % of 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib) or a pharmaceutically-acceptable salt thereof;
(ii) about 20 to about 50 wt % of lactose monohydrate; and
(iii) about 15 to about 50 wt % of microcrystalline cellulose.

The invention further provides a pharmaceutical composition comprising:
(i) about 10 to about 40 wt % 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib) or a pharmaceutically-acceptable salt thereof; and
(ii) about 0.2 to about 5 wt % hydrophobic colloidal silica.

The invention further provides a pharmaceutical composition comprising:
(i) about 10 to about 40 wt % 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib) or a pharmaceutically-acceptable salt thereof; and
(ii) about 0.5 to about 5 wt % of sodium starch glycolate.

The invention also provides solid oral dosage forms of the pharmaceutical compositions defined above, in particular tablets. The tablets may comprise a tablet core comprising the pharmaceutical compositions of the invention wherein the tablet cores are provided with a coating, e.g., to make the tablets easier to swallow and to enhance the visual appearance of the tablets. The tablet cores and coated tablets of the invention are found to exhibit simultaneously the desirable characteristics of exceptional physical stability, high tablet hardness and high core strength, rapid dissolution and high bioavailability.

The invention further provides a method of preparing tablets comprising brigatinib, wherein the method comprises the steps of:
(i) blending brigatinib with one or more of lactose monohydrate, microcrystalline cellulose, hydrophobic colloidal silica, sodium starch glycolate, and magnesium stearate so as to obtain a pharmaceutical composition according to any of the first, second or third aspects of the invention; and
(ii) compressing the blended pharmaceutical composition to form a tablet core.

The invention further provides a method of preparing tablets comprising brigatinib, wherein the method comprises the steps of:
(i) blending brigatinib or a pharmaceutically-acceptable salt thereof with one or more of lactose monohydrate, microcrystalline cellulose, hydrophobic colloidal 5 silica, sodium starch glycolate, and magnesium stearate so as to obtain a pharmaceutical composition according to any of the first, second or third aspects of the invention; and
(ii) compressing the blended pharmaceutical composition to form a tablet core.

The method may optionally further comprise coating the tablet cores with a coating, which may be selected from polymeric coatings, such as polysaccharides, PVA (polyvinyl alcohol) and acrylics. The brigatinib-containing compositions of the invention have the further advantage that they may be used in accordance with the method of the invention to manufacture brigatinib-containing tablet cores without an unacceptable frequency of defects.

The invention further provides a method of treating a disease or disorder responsive to the inhibition of ALK (such as non-small cell lung cancer) comprising administering a pharmaceutical composition as described herein to a patient in need of such treatment.

The invention further provides a pharmaceutical composition as described herein for use in a method of treating a disease or disorder responsive to the inhibition of ALK (such as non-small cell lung cancer), the method comprising administering the pharmaceutical composition to a patient in need of such treatment.

### BRIEF DESCRIPTION OF THE FIGURES

FIG. 1 shows a representative co-processing process using brigatinib and colloidal silicon dioxide.

### DETAILED DESCRIPTION OF THE INVENTION

It has been found that pharmaceutical formulations comprising brigatinib are highly and unusually sensitive to the choice of excipients used. Following extensive studies by the applicant, it has been found that the stability of the brigatinib drug substance as well as the ability to manufacture brigatinib-containing tablets with a high level of strength and hardness has been found to depend closely on the excipients selected. Even when suitable excipients have been identified, it is found that brigatinib has relatively poor compaction properties and therefore pharmaceutical compositions comprising brigatinib have a relatively narrow compressibility window if problems of poor cohesion and friability are to be avoided. The inventors have also found that specific pharmaceutical formulations and manufacturing methods are necessary for optimum performance because brigatinib can be highly and unusually cohesive.

To address these problems, the applicant has developed optimized pharmaceutical compositions comprising brigatinib.

As used herein, the term “pharmaceutical composition” refers to a composition comprising a specified amount of an active pharmaceutical ingredient and one or more pharmaceutically acceptable excipients, suitable for administration to a human or other mammal subject. The pharmaceutical compositions of the invention are preferably dry compositions in which the components of the composition are present in a particulate (e.g. powder or granular) form. The components of the composition are typically suitably blended to form a substantially homogenous composition. Excipients identified herein suitably comply with the specifications for pharmaceutical use as set out in one or more of the United States Pharmacopeia, National Formulary, European Pharmacopeia and Japanese Pharmacopeia.

As used herein, the term “excipient” refers to a pharmaceutically acceptable ingredient, other than an active pharmaceutical ingredient, that is used to formulate an active pharmaceutical ingredient for administration to a patient. Categories of excipients commonly used in the pharmaceutical industry for the preparation of solid dosage forms include fillers, binders, lubricants, glidants, disintegrants and preservatives. The choice of excipients within each category, the amounts thereof, and their degree of compatibility with the active pharmaceutical ingredient gives rise to an extremely wide range of possible formulations of widely varying properties.

In a first aspect, the invention provides a pharmaceutical composition comprising:
(i) about 10 to about 40 wt % of 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib);
(ii) about 20 to about 50 wt % of lactose monohydrate; and
(iii) about 15 to about 50 wt % of microcrystalline cellulose.

In a first aspect, the invention provides a pharmaceutical composition comprising:
(i) about 10 to about 40 wt % of 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib) or a pharmaceutically-acceptable salt thereof;
(ii) about 20 to about 50 wt % of lactose monohydrate; and
(iii) about 15 to about 50 wt % of microcrystalline cellulose.

Lactose monohydrate and microcrystalline cellulose are used as fillers in the pharmaceutical compositions of the invention, and it has been found that the use of lactose monohydrate and microcrystalline cellulose as fillers (both individually and in combination) results in increased stability of the brigatinib active ingredient when compared to other fillers that are available in the art.

The pharmaceutical composition of the first aspect of the invention preferably comprises one or more glidants. More preferably, the pharmaceutical composition of the first aspect of the invention comprises hydrophobic colloidal silica. Still more preferably, the pharmaceutical composition of the first aspect of the invention comprises about 0.2 to about 3 wt % of hydrophobic colloidal silica. The hydrophobic colloidal silica may be used as a glidant in order to address problems caused by cohesiveness of the brigatinib in the composition. In order to effectively enhance the flowability of brigatinib particles, the hydrophobic colloidal silica preferably forms an adherent coating on the surfaces of the brigatinib particles, thus providing the brigatinib surface with a less cohesive or sticky outer surface that facilitates the formation of homogenous blended compositions comprising the brigatinib particles, and that prevents manufacturing problems due to sticking of the pharmaceutical composition to die walls during the formation of tablet cores by compression. An “adherent coating” is a coating adhered to brigatinib particles and at least partially covering the surface of the brigatinib particles. Optimized methods of combining the brigatinib drug substance and the hydrophobic colloidal silica as described herein may be used to further enhance the performance of the compositions of the invention.

The pharmaceutical composition of the first aspect of the invention preferably comprises one or more disintegrants. Disintegrants are substances that expand upon contact with moisture in the digestive tract and thus facilitate the disintegration of tablets and the release of the brigatinib active ingredient following ingestion. A preferred disintegrant is sodium starch glycolate Type A. Preferably, sodium starch glycolate Type A is present in an amount of from about 0.5 to about 5 wt % of the pharmaceutical composition. It has been found that the use of sodium starch glycolate Type A as a disintegrant results in improved stability of the brigatinib active ingredient when compared to other disintegrants that are available in the art.

In a second aspect, the invention provides a pharmaceutical composition comprising:
(i) about 10 to about 40 wt % 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib); and
(ii) about 0.2 to about 3 wt % hydrophobic colloidal silica.

In a second aspect, the invention provides a pharmaceutical composition comprising:
(i) about 10 to about 40 wt % 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib) or a pharmaceutically-acceptable salt thereof; and
(ii) about 0.2 to about 3 wt % hydrophobic colloidal silica.

In accordance with the second aspect of the invention, the hydrophobic colloidal silica preferably forms an adherent coating on the surfaces of brigatinib particles. Optimized methods of combining the brigatinib drug substance and the hydrophobic colloidal silica as described herein may be used to further enhance the performance of the compositions of the invention.

The pharmaceutical composition of the second aspect of the invention preferably comprises one or more fillers. More preferably, the pharmaceutical composition of the second aspect of the invention comprises one or more of lactose monohydrate and microcrystalline cellulose. Still more preferably, the pharmaceutical composition of the second aspect of the invention comprises about 20 to about 50 wt % of lactose monohydrate and about 15 to about 50 wt % of microcrystalline cellulose.

The pharmaceutical composition of the second aspect of the invention preferably comprises one or more disintegrants. More preferably, the pharmaceutical composition of the second aspect of the invention comprises sodium starch glycolate Type A. Still more preferably, the pharmaceutical composition of the second aspect of the invention comprises about 0.5 to about 5 wt % of sodium starch glycolate Type A.

In a third aspect, the invention provides a pharmaceutical composition comprising:
(i) about 10 to about 40 wt % 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib); and
(ii) about 0.5 to about 5 wt % sodium starch glycolate Type A.

In a third aspect, the invention provides a pharmaceutical composition comprising:
(i) about 10 to about 40 wt % 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib) or a pharmaceutically-acceptable salt thereof; and
(ii) about 0.5 to about 5 wt % sodium starch glycolate Type A.

It has been found that the use of sodium starch glycolate Type A as a disintegrant results in improved stability of the brigatinib active ingredient when compared to other disintegrants that are available in the art.

The pharmaceutical composition of the third aspect of the invention preferably comprises one or more fillers. More preferably, the pharmaceutical composition of the third aspect of the invention comprises one or more of lactose monohydrate and microcrystalline cellulose. Still more preferably, the pharmaceutical composition of the third aspect of the invention comprises about 20 to about 50 wt % of lactose monohydrate and about 15 to about 50 wt % of microcrystalline cellulose.

The pharmaceutical composition of the third aspect of the invention preferably comprises one or more glidants. More preferably, the pharmaceutical composition of the third aspect of the invention comprises hydrophobic colloidal silica. Still more preferably, the pharmaceutical composition of the third aspect of the invention comprises about 0.2 to about 3 wt % of hydrophobic colloidal silica, wherein the hydrophobic colloidal silica preferably forms an adherent coating on the surfaces of brigatinib particles. Optimized methods of combining the brigatinib drug substance and the hydrophobic colloidal silica described herein may be used to further enhance the performance of the compositions of the invention.

The pharmaceutical compositions of the invention preferably comprise brigatinib or a pharmaceutically-acceptable salt thereof in an optimized amount of from about 12 to about 35 wt %, more preferably about 15 to about 30 wt % and most preferably about 18 to about 25 wt % based on the total weight of the pharmaceutical composition. It has been found that the use of brigatinib in these optimized amounts together with the specific choice of excipients identified herein provides an effective solution to the friability problems of brigatinib-containing compositions.

The pharmaceutical compositions of the invention preferably comprise lactose monohydrate in an optimized amount of from about 25 to about 45 wt %, more preferably about 30 to about 40 wt % and most preferably about 32 to about 38 wt % based on the total weight of the pharmaceutical composition.

The pharmaceutical compositions of the invention preferably comprise microcrystalline cellulose in an optimized amount of from about 20 to about 45 wt %, more preferably about 25 to about 40 wt %, more preferably from about 30 to about 40 wt % and most preferably about 32 to about 38 wt % based on the total weight of the pharmaceutical composition.

The pharmaceutical compositions of the invention preferably comprise hydrophobic colloidal silica in an optimized amount of from about 0.4 to about 2 wt %, more preferably from about 0.6 to about 1.5 wt %, and most preferably from about 0.8 to about 1.2 wt %. As noted above, the hydrophobic colloidal silica preferably forms an adherent coating on the surfaces of brigatinib particles. Brigatinib particles with an adherent coating of hydrophobic colloidal silica may be obtained by blending brigatinib particles with hydrophobic colloidal silica, e.g., prior to the addition of other components of the pharmaceutical compositions of the invention.

The brigatinib particles with an adherent coating of hydrophobic colloidal silica are preferably obtained by blending brigatinib and hydrophobic colloidal silica and passing the blended mixture of brigatinib and hydrophobic colloidal silica through a screening mill having a screen size in the range of from 400 to 800 μm. The mixture of brigatinib and hydrophobic colloidal silica is preferably passed through the screening mill several times, preferably from 2 to 50 times, or from 5 to 20 times, for example 10 times, so as to obtain optimized distribution of hydrophobic colloidal silica over the brigatinib surface and optimized flowability and dispersibility of brigatinib in the compositions of the invention.

The pharmaceutical compositions of the invention preferably comprise sodium starch glycolate Type A in an optimized amount of from about 1 to about 5 wt %, more preferably about 1.5 to about 4.5 wt %, and more preferably about 2 to about 4 wt %.

In order to enhance the manufacturability of solid dosage forms, particularly tablets, comprising the pharmaceutical composition, the composition of the first aspect of the invention preferably further comprises one or more lubricants. The use of lubricants prevents sticking of the pharmaceutical composition to die walls during compression and ejection of tablet cores. A preferred lubricant is magnesium stearate. Suitably, the magnesium stearate is present in an amount of from about 0.2 to about 3 wt %, for example from about 0.5 to about 2.5 wt %, from about 0.8 to about 2 wt % or from about 1 to about 1.8 wt %.

Brigatinib may be in the free base form or in the form of a pharmaceutically-acceptable salt of brigatinib. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines are well known in the art. For example, S. M. Berge, et al. describe pharmaceutically acceptable salts in detail in J. Pharmaceutical Sciences, 66: 1-19 (1977), incorporated herein by reference. Salts of brigatinib can be prepared in situ during the isolation and purification of brigatinib, or separately by reacting the free base of brigatinib with a suitable acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Preferably, brigatinib is in the free base form. References herein to 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine or to brigatinib shall be taken to mean the free base form of brigatinib unless specified otherwise.

A preferred pharmaceutical composition according to the invention comprises:
(i) about 10 to about 40 wt % of 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib);
(ii) about 20 to about 50 wt % of lactose monohydrate;
(iii) about 15 to about 50 wt % of microcrystalline cellulose;
(iv) about 0.5 to about 5 wt % of sodium starch glycolate Type A;
(v) about 0.2 to about 2 wt % of hydrophobic colloidal silica;
(vi) about 0.2 to about 3 wt % of magnesium stearate.

In some embodiments, the composition consists entirely of components (i)-(vi).

A further preferred pharmaceutical composition according to the invention comprises:
(i) about 12 to about 35 wt % of 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib);
(ii) about 25 to about 45 wt % of lactose monohydrate;
(iii) about 20 to about 45 wt % of microcrystalline cellulose;
(iv) about 1 to about 5 wt % of sodium starch glycolate Type A;
(v) about 0.4 to about 1.8 wt % of hydrophobic colloidal silica;
(vi) about 0.5 to about 2.5 wt % of magnesium stearate.

In some embodiments, the composition consists entirely of components (i)-(vi).

A further preferred pharmaceutical composition according to the invention comprises:
(i) about 15 to about 30 wt % of 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib);
(ii) about 30 to about 40 wt % of lactose monohydrate;
(iii) about 25 to about 40 wt % of microcrystalline cellulose;
(iv) about 1.5 to about 4.5 wt % of sodium starch glycolate Type A;
(v) about 0.6 to about 1.5 wt % of hydrophobic colloidal silica;
(vi) about 0.8 to about 2 wt % of magnesium stearate.

In some embodiments, the composition consists entirely of components (i)-(vi).

A further preferred pharmaceutical composition according to the invention comprises:
(i) about 18 to about 25 wt % of 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib);
(ii) about 32 to about 38 wt % of lactose monohydrate;
(iii) about 30 to about 38 wt % of microcrystalline cellulose;
(iv) about 2 to about 4 wt % of sodium starch glycolate Type A;
(v) about 0.8 to about 1.2 wt % of hydrophobic colloidal silica;
(vi) about 1 to about 1.8 wt % of magnesium stearate.

In some embodiments, the composition consists entirely of components (i)-(vi).

A particularly preferred pharmaceutical composition according to the invention consists of:
(i) about 20 wt % of 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib);
(ii) about 37 to about 38 wt % of lactose monohydrate;
(iii) about 37 to about 38 wt % of microcrystalline cellulose;
(iv) about 3 wt % of sodium starch glycolate Type A;
(v) about 1 wt % of hydrophobic colloidal silica;
(vi) about 1.25 wt % of magnesium stearate.

The present invention provides an optimized brigatinib-containing pharmaceutical composition for the preparation of solid oral forms of brigatinib and it will be understood that the incorporation of additional excipients other than those specifically identified above may have a deleterious effect on the properties of the composition, for instance in terms of the stability of the brigatinib drug substance, or the manufacturability of solid oral dosage forms comprising the pharmaceutical compositions of the invention. Accordingly, the amount of any additional excipients other than those specifically identified above is preferably less than about 10 wt % of the pharmaceutical composition, more preferably less than about 5 wt % of the composition, more preferably less than about 2 wt % of the composition, more preferably less than about 1 wt % of the composition, and most preferably less than about 0.5 wt % of the composition. Optimally, the pharmaceutical compositions of the invention may consist only of those excipients specifically identified above, in the proportions indicated. Preferably, the pharmaceutical compositions of the invention do not comprise dibasic calcium phosphate, croscarmellose sodium or sodium lauryl sulfate.

Brigatinib may exist in a number of polymorphic forms, designated as Forms A to K, as described in detail in WO 2016/065028. In the pharmaceutical compositions of the present invention, the brigatinib preferably comprises brigatinib Form A. For example, the compositions of the invention can comprise at least about 50 wt % of brigatinib Form A, based on the total amount of brigatinib. In some embodiments, the brigatinib may comprise at least about 60 wt % of brigatinib Form A, based on the total amount of brigatinib. In some embodiments, the brigatinib may comprise at least about 70 wt % of brigatinib Form A. In some embodiments, the brigatinib may comprise at least about 80 wt % of brigatinib Form A. In some embodiments, the brigatinib may comprise at least about 90 wt % of brigatinib Form A. In some embodiments, the brigatinib may comprise at least about 95 wt % of brigatinib Form A. In some embodiments, the brigatinib may comprise at least about 98 wt % of brigatinib Form A. In some embodiments, the brigatinib may comprise at least about 99 wt % of brigatinib Form A. Suitably, the brigatinib may consist entirely of brigatinib Form A.

Brigatinib Form A is anhydrous and non-hygroscopic and does not convert to other polymorphic forms via solvent-mediated or solid-solid transitions or by exposure to elevated temperature, elevated humidity, mechanical pressure or grinding. The chemical and crystal structures of brigatinib Form A have been established unambiguously by a combination of NMR spectroscopy, mass spectroscopy, X-ray powder diffraction and single crystal X-ray crystallography. Confirmatory data is provided by elemental analysis and FT-IR spectroscopy. The pharmaceutical compositions of the present invention are particularly suitable for formulating brigatinib Form A because Form A is particularly and unusually cohesive, often due to the particles of Form A having plate-like morphology.

Throughout the specification including each embodiment, the total weight % of the pharmaceutical composition is about 100% (excluding coatings).

When the term “about” is used in conjunction with a numerical value or range, it modifies that value or range by extending the boundaries above and below those numerical value(s). In general, the term “about” is used herein to modify a numerical value above and below the stated value by a variance of 10%, 5%, or 1%. In some embodiments, the term “about” is used to modify a numerical value above and below the stated value by a variance of 10%. In some embodiments, the term “about” is used to modify a numerical value above and below the stated value by a variance of 5%. In some embodiments, the term “about” is used to modify a numerical value above and below the stated value by a variance of 1%.

In accordance with the invention, the brigatinib particle size may be controlled in order to optimize the properties of solid oral dosage forms comprising the pharmaceutical composition of the invention. It has been found that increased hardness and reduced friability of tablet cores comprising the pharmaceutical composition are obtained when the brigatinib has a D₅₀ particle size in the range of from about 5 to about 25 μm, preferably from about 6 to about 25 μm, preferably from about 8 to about 22 μm, more preferably from about 10 to about 20 μm.

The D₁₀ particle size of the brigatinib particles is preferably at least 0.5 μm, more preferably at least 1 μm, more preferably at least about 1.5 μm, more preferably at least about 2 μm, more preferably at least about 2.5 μm, but no more than about 8.0 μm.

The D₉₀ particle size of the brigatinib particles is preferably no more than about 90 μm, more preferably no more than about 60 μm, more preferably no more than about 55 μm, more preferably no more than about 50 μm, more preferably no more than about 45 μm.

More particularly, it has been found that improved flowability of brigatinib and thus increased homogeneity of the blended pharmaceutical composition, as well as increased hardness and reduced friability of tablet cores comprising the pharmaceutical composition, are obtained when the brigatinib has:
(a) a D₅₀ particle size in the range of from 5 to 25 μm, preferably from 6 to 15 μm, more preferably from 8 to 10 μm; and/or
(b) a D₁₀ particle size of at least 1 μm, more preferably at least 1.5 μm, more preferably at least 1.8 μm, for example at least 2 μm, or at least 2.5 μm; and/or
(c) a D₉₀ particle size of no more than 40 μm, more preferably no more than 35 μm, more preferably no more than 30 μm, more preferably no more than 25 μm.

In a more preferred embodiment, the brigatinib has a D₅₀ particle size in the range of from 6 to 15 μm, a D₁₀ particle size of at least 1.5 μm, and a D₉₀ particle size of no more than 30 μm.

In a particularly preferred embodiment, the brigatinib has a D₅₀ particle size in the range of from 8 to 10 μm, a D₁₀ particle size of at least 1.8 μm, and a D₉₀ particle size of no more than 25 μm.

The term “particle size” as used herein refers to the equivalent spherical diameter (esd), i.e. the diameter of a sphere having the same volume as a given particle. The terms “D₅₀” and “D₅₀ particle size” as used herein refer to the volume-based median particle diameter, i.e. the diameter below which about 50% by volume of the particle population is found. The terms “D₁₀” and “D₁₀ particle diameter” as used herein refer to the 10th percentile volume-based median particle diameter, i.e. the diameter below which about 10% by volume of the particle population is found. The terms “D₉₀” and “D₅₀ particle diameter” as used herein refer to the 90th percentile volume-based median particle diameter, i.e. the diameter below which about 90% by volume of the particle population is found.

Particle diameters and particle size distributions as reported herein can be determined by routine laser diffraction techniques. Laser diffraction relies on the principle that a particle will scatter light at an angle that varies depending on the size the particle and a collection of particles will produce a pattern of scattered light defined by intensity and angle that can be correlated to a particle size distribution. A number of laser diffraction instruments are commercially available for the rapid and reliable determination of particle size distributions. Unless stated otherwise, particle size distribution measurements as specified or reported herein are as measured using a Beckman Coulter LS 13 320 Laser Diffraction Particle Sizer.

The pharmaceutical composition of the invention is preferably storage stable for at least 6 months at about 25° C. and about 60% relative humidity, wherein storage stability may be defined as the formation of no more than about 2%, preferably no more than 1%, by weight of brigatinib-related impurities based on the initial amount of brigatinib, as determined by HPLC. Preferably, the pharmaceutical composition of the invention is storage stable for at least 8 weeks at about 40° C. and about 75% relative humidity and/or for at least 8 weeks at about 60° C. and ambient humidity.

The pharmaceutical compositions of the invention are preferably in a solid oral dosage form. The oral solid dosage form includes tablets, pills, capsules, powders. Preferably, the solid oral dosage form is a tablet.

In a fourth aspect, the invention provides tablets comprising a tablet core comprising or consisting of a pharmaceutical composition as defined above and optionally a coating.

Suitable coatings may be selected from polymeric coatings and sugar coatings. The coatings are typically applied in order to achieve a weight gain of from about 0.5 to about 10 wt %, preferably about 1 to about 8 wt %, preferably about 2 to about 5 wt % based on 100 wt % of the tablet core. Typically, the coating thickness is in the range of from about 20 to about 100 μm. The coating may comprise one or more additives to enhance the properties of the tablets or to facilitate the coating process, e.g. pigments, plasticizers and surfactants.

Examples of polymers which may be used as coatings for tablets according to the invention include cellulose derivatives, such as cellulose ethers, acrylic polymers and copolymers, methacrylic polymers and copolymers polyethylene glycols, polyvinyl pyrrolidones, and polyvinyl alcohols. Examples of suitable coating polymers include methyl cellulose, ethyl cellulose, hydroxyethyl cellulose, hydroxypropyl cellulose, hydroxypropyl methyl cellulose, hydroxypropyl ethyl cellulose, polyvinyl pyrrolidone polyvinyl acetate, Copovidone, hydroxypropylmethyl cellulose acetate succinate (HPMC AS) and hydroxypropylmethyl cellulose phthalate (HPMCP). A preferred coating polymer is PVA, for example PVA-based coatings as marketed under the “Opadry” brand by Colorcon.

The tablet and any coating are preferably selected for immediate release of the brigatinib drug substance following ingestion of the tablets by a patient. As used herein the term “immediate-release” has its conventional meaning in the art. For example, an immediate release composition typically provides rapid release of the majority of the therapeutic compound, for example the release of at least about 60%, at least about 70%, at least about 80% or at least about 90% of the brigatinib drug substance within a period of e.g. 30 minutes following oral ingestion.

The tablets of the invention may suitably comprise one or more identifying markers. For instance the tablets may be embossed or debossed with an identifying marker or an identifying marker may be printed onto the surface of the tablets.

The tablets of the invention may suitably comprise from about 5 to about 500 mg brigatinib, preferably from about 10 to about 250 mg brigatinib, and more preferably from about 20 to about 200 mg brigatinib. For example the tablets of the invention may comprise about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg or about 200 mg of brigatinib. In a preferred embodiment, the tablets of the invention may comprise about 30 mg of brigatinib. In another preferred embodiment, the tablets of the invention may comprise about 60 mg of brigatinib. In another preferred embodiment, the tablets of the invention may comprise about about 90 mg of brigatinib. In another preferred embodiment, the tablets of the invention may comprise about 180 mg of brigatinib. The loading of brigatinib may be less than about 30 wt % of the tablet core, preferably less than about 25 wt % of the tablet core. In some embodiments, the loading of brigatinib may be about about 20 wt % of the tablet core. In a preferred embodiment, the tablets of the invention may comprise about 30 mg, about 90 mg or about 180 mg of brigatinib at an about 20 wt % loading of brigatinib in the tablet core. Where brigatinib is in the form of a pharmaceutically-acceptable salt, the above drug loadings are based on the amount of brigatinib free base and do not take into account the weight of the acid used to form the salt.

The tablets may be round or lozenge-shaped. Lozenge-shaped tablets are preferred for tablets comprising higher doses of brigatinib (e.g. about 90 mg or about 180 mg of brigatinib at an about 20 wt % loading of brigatinib) as they may be swallowed more easily by patients.

In a fifth aspect, the invention provides a method of preparing tablets comprising brigatinib, wherein the method comprises the steps of:
(i) blending brigatinib with one or more of lactose monohydrate, microcrystalline cellulose, hydrophobic colloidal silica, sodium starch glycolate, and magnesium stearate so as to obtain a pharmaceutical composition according to any of the first, second or third aspects of the invention; and
(ii) compressing the blended pharmaceutical composition to form a tablet core.

In a fifth aspect, the invention provides a method of preparing tablets comprising brigatinib, wherein the method comprises the steps of:
(i) blending brigatinib or a pharmaceutically-acceptable salt thereof with one or more of lactose monohydrate, microcrystalline cellulose, hydrophobic colloidal silica, sodium starch glycolate, and magnesium stearate so as to obtain a pharmaceutical composition according to any of the first, second or third aspects of the invention; and
(ii) compressing the blended pharmaceutical composition to form a tablet core.

It has surprisingly been found that the pharmaceutical compositions of the invention may be supplied to a direct compression process to yield tablets meeting desirable specifications for strength, hardness and content uniformity without the need for conventional wet or dry granulation steps or wet milling. Thus, in accordance with the invention, the method defined above preferably does not include at least one of wet granulation, dry granulation and wet milling. More preferably, the method of the invention does not comprise any of wet granulation, dry granulation and wet milling.

The brigatinib in step (i) is preferably in the free-base form.

In a preferred embodiment, step (i) of the method of the invention comprises the step of:
(ia) blending brigatinib and hydrophobic colloidal silica and passing the blended mixture of brigatinib and hydrophobic colloidal silica through a screening mill having a screen size in the range of from about 400 to about 800 μm.

The mixture of brigatinib and hydrophobic colloidal silica is preferably passed through the screening mill several times, preferably from 2 to 50 times, more preferably from 5 to 20 times, for example 10 times.

It has been found that repeated screening of the mixture of brigatinib and hydrophobic colloidal silica according to the method of the invention results is a key factor in obtaining effective distribution of the hydrophobic colloidal silica over the surfaces of the brigatinib particles. Brigatinib particles having an adherent coating of hydrophobic colloidal silica formed by the repeated screening method of the invention provide a substantial reduction in agglomeration of the brigatinib particles when compared to conventional methods of blending active pharmaceutical ingredients with excipients. The method of the invention therefore provides increased homogeneity of the blended pharmaceutical composition together with increased hardness and reduced friability of the tablet cores.

A further improvement in these properties is obtained when step (i)/(ia) is carried out using brigatinib having:
(a) a D₅₀ particle size in the range of from 5 to 25 μm, preferably from 6 to 15 μm, more preferably from 8 to 10 μm; and/or
(b) a D₁₀ particle size of at least 1 μm, more preferably at least 1.5 μm, more preferably at least 1.8 μm, for example at least 2 μm or at least 2.5 μm; and/or
(c) a D₉₀ particle size of no more than 40 μm, more preferably no more than 35 μm, more preferably no more than 30 μm, more preferably no more than 25 μm.

In order to obtain brigatinib having D₁₀, D₅₀ and D₉₀ values within the preferred ranges set out above, the inventors have developed a novel crystallisation process. In a preferred embodiment, the brigatinib used in step (i)/(ia) is prepared by forming a solution of brigatinib in a mixture of 1-propanol and ethyl acetate at 70-90° C., adding seed crystals of brigatinib, and cooling the mixture at a rate of 10-20° C./hour to 0±5° C. for up to 30 hours, followed by separation of the brigatinib crystals from the crystallisation mother liquor.

1-propanol and ethyl acetate are suitably used in a volume ratio of from 5:1 to 1:1, for example from 4:1 to 2:1 and preferably about 3:1.

The brigatinib seed crystals are preferably used in an amount of from 0.001 to 0.01 wt % based on the amount of brigatinib in solution. The brigatinib seed crystals may be crystals of brigatinib polymorphic Form A.

The mixture of 1-propanol and ethyl acetate is suitably used in an amount of from 2 to 10 parts by weight, more preferably 3 to 7 parts by weight, more preferably 4 to 6 parts by weight, for example 5 parts by weight, per 1 part by weight of brigatinib in solution.

In a preferred embodiment, step (i) of the method of the invention comprises the step of:
(ib) blending the mixture from step (ia) with one or more of lactose monohydrate, microcrystalline cellulose, sodium starch glycolate, and magnesium stearate.

The pharmaceutical composition may be compressed in step (ii) to form a tablet core using a rotary tablet press. The rotary tablet press is provided with tooling appropriate to the size of the tablet that is required and the tablet dies and/or presses may be embossed or debossed with suitable identifying markings. The compression parameters are suitably selected so as to obtain tablets having a hardness in the range of from 10 to 20 kg-force.

The tablets prepared according to the method of the invention may suitably comprise from about 5 to about 500 mg brigatinib, preferably from about 10 to about 250 mg brigatinib, and more preferably from about 20 to about 200 mg brigatinib. For example the tablets of the invention may comprise about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg or about 200 mg of brigatinib. In a preferred embodiment, the tablets prepared according to the invention may comprise about 30 mg of brigatinib. In another preferred embodiment, the tablets prepared according to the invention may comprise about 60 mg of brigatinib. In another preferred embodiment, the tablets prepared according to the invention may comprise about 90 mg of brigatinib. In another preferred embodiment, the tablets prepared according to the invention may comprise about 180 mg of brigatinib. The loading of brigatinib may be less than about 30 wt % of the tablet core, preferably less than about 25 wt %. In some embodiments, the loading of brigatinib may be about 20 wt % of the tablet core. In a preferred embodiment, the tablets of the invention may comprise about 30 mg, about 90 mg or about 180 mg of brigatinib at an about 20 wt % loading of brigatinib in the tablet core. Where brigatinib is in the form of a pharmaceutically-acceptable salt, the above drug loadings are based on the amount of brigatinib free base and do not take into account the weight of the acid used to form the salt.

The method of the invention may optionally further comprise the step of:
(iii) providing the tablet core with a polymeric coating.

Suitable polymeric coating types are defined above. The polymeric coating is suitably provided in an amount effective to obtain a dry weight gain of from about 0.5 to about 10 wt %, preferably about 1 to about 8 wt %, preferably about 2 to about 5 wt % based on about 100 wt % of the tablet core.

Coating of the tablets in step (iii) is typically carried out as a batch process inside a perforated rotating coating pan. As a bed of tablet cores are continually agitated, a liquid solution or suspension of the coating polymer and any additives is sprayed onto the tablet cores. A flow of heated air drawn through the tablet bed dries the coating solution/suspension so as to provide the tablet cores with an even amount of dried coating.

The invention provides tablets obtainable by the method of the fifth aspect of the invention.

The pharmaceutical compositions and tablets described herein may be used for the treatment of diseases/disorders that are responsive to the inhibition of ALK, in particular for the treatment of cancer.

In a sixth aspect, the invention therefore provides a method of treating a disease or disorder responsive to the inhibition of ALK, the method comprising administering a pharmaceutical composition as defined above to a patient in need of such treatment. Suitably, the pharmaceutical composition is in the form of a tablet according to the fourth aspect of the invention.

In a seventh aspect, the invention provides a pharmaceutical composition as defined above for use in a method of treating a disease or disorder responsive to the inhibition of ALK, the method comprising administering a pharmaceutical composition as defined above to a patient in need of such treatment. Suitably, the pharmaceutical composition is in the form of a tablet according to the fourth aspect of the invention.

In some embodiments, the disease or disorder responsive to the inhibition of ALK in an ALK+ driven cancer, such as non-small cell lung cancer, in particular ALK-positive non-small cell lung cancer. The ALK-positive non-small cell lung cancer may be locally advanced or metastatic ALK-positive non-small cell lung cancer.

The pharmaceutical compositions of the invention may also be effective for the treatment of other cancers. Such cancers include, but are not limited to, cancers of the breast, neural tumors such as glioblastomas and neuroblastomas; esophageal carcinomas, soft tissue cancers such as rhabdomyosarcomas, among others; various forms of lymphoma such as a non-Hodgkin's lymphoma (NHL) known as anaplastic large-cell lymphoma (ALCL), various forms of leukemia; and including cancers which are ALK or c-met mediated.

In some embodiments, the patient has previously been treated with crizotinib or another tyrosine kinase inhibitor.

The pharmaceutical compositions of the invention are administered to patients in an amount effective to inhibit the growth or spread of cancer cells, the size or number of tumours, or to obtain some other measurable benefit in terms of the level, stage, progression or severity of the cancer. The exact amount required may depend on factors including the age and condition of the patient, the severity of the disease, and the use of other therapeutically active substances in combination with the pharmaceutical compositions of the invention. In one embodiment, the pharmaceutical compositions of the invention may be administered to patients as a single dose of about 180 mg brigatinib per day. In another embodiment, the pharmaceutical compositions of the invention may be administered to patients as a single dose of about 90 mg brigatinib per day for seven days, followed by a single dose of about 180 mg brigatinib per day.

Pharmaceutical compositions as disclosed herein can be administered as part of a treatment regimen in which brigatinib is the sole active pharmaceutical agent, or used in combination with one or more other therapeutic agents as part of a combination therapy. When administered as one component of a combination therapy, the therapeutic agents being administered can be formulated as separate compositions that are administered at the same time or sequentially at different times (e.g., within 72 hours, 48 hours, or 24 hours of one another).

Thus, the administration of brigatinib in a pharmaceutical composition as disclosed herein can be in conjunction with at least one additional therapeutic agent known to those skilled in the art in the prevention or treatment of cancer, such as radiation therapy or cytostatic agents, cytotoxic agents, other anti-cancer agents and other drugs to ameliorate symptoms of the cancer or side effects of any of the drugs. Non-limiting examples additional therapeutic agents include agents suitable for immunotherapy (such as, for example, PD-1 and PDL-1 inhibitors), antiangiogenesis (such as, for example, bevacizumab), and/or chemotherapy. A comprehensive list of therapeutic agents which may be used in combination therapies with the pharmaceutical compositions of the invention may be found in WO 2016/065028.

The various aspects and embodiments of the invention described herein can be combined.

In further aspects, the invention provides pharmaceutical compositions, methods and uses as defined above except that lactose monohydrate is replaced by anhydrous lactose. In these aspects of the invention, anhydrous lactose may be used in the same weight percentages as are specified above for lactose monohydrate, and all other features of the pharmaceutical compositions, methods and uses are unchanged from those defined above.

The invention further provides a method of crystallizing brigatinib comprising forming a solution of brigatinib in a mixture of 1-propanol and ethyl acetate at 70-90° C., adding seed crystals of brigatinib, and cooling the mixture at a rate of 10-20° C./hour to 0±5° C. for up to 30 hours, followed by separation of the brigatinib crystals from the crystallisation mother liquor.

In accordance with the method of the invention, 1-propanol and ethyl acetate are preferably used in a volume ratio of from 5:1 to 1:1, for example from 4:1 to 2:1 and preferably about 3:1.

The brigatinib seed crystals are preferably used in an amount of from 0.001 to 0.01 wt % based on the amount of brigatinib in solution. The brigatinib seed crystals may be crystals of brigatinib polymorphic Form A.

The mixture of 1-propanol and ethyl acetate is suitably used in an amount of from 2 to 10 parts by weight, more preferably 3 to 7 parts by weight, more preferably 4 to 6 parts by weight, for example 5 parts by weight, per 1 part by weight of brigatinib in solution.

The invention further provides crystalline brigatinib obtainable by the crystallization method described above.

Preferably, the crystalline brigatinib obtained according to the crystallization method of the invention has the brigatinib has:
(a) a D₅₀ particle size in the range of from 5 to 25 μm, preferably from 6 to 15 μm, more preferably from 8 to 10 μm; and/or
(b) a D₁₀ particle size of at least 1 μm, more preferably at least 1.5 μm, more preferably at least 1.8 μm, for example at least 2 μm, or at least 2.5 μm; and/or
(c) a D₉₀ particle size of no more than 40 μm, more preferably no more than 35 μm, more preferably no more than 30 μm, more preferably no more than 25 μm.

### EXAMPLES

### Example 1—Preparation of Tablets Comprising a Pharmaceutical Composition According to the Invention

A typical process for the preparation of brigatinib-containing tablets in accordance with the invention is described below.

Brigatinib drug substance (20 parts by weight, polymorphic Form A, D₅₀=9.6 μm, D₁₀=2.7 μm, D₅₀=23.1 μm) and hydrophobic colloidal silica (1 part by weight) were weighed and sieved before being added to an intermediate container blender. The mixture was blended until a substantially homogenous mixture was obtained (typically 125 to 375 revolutions at 15 rpm). Milling and screening of the blended mixture was carried out by passing the mixture ten times through a screening mill having a screen size of 610 μm.

Lactose monohydrate (37.37 parts by weight), microcrystalline cellulose (37.38 parts by weight) and sodium starch glycolate (Type A, 3 parts by weight) were weighed and sieved and added to the blended mixture of brigatinib and hydrophobic colloidal silica and further blended until a substantially homogenous mixture was obtained (typically 250 to 500 revolutions at 15 rpm).

Magnesium stearate (1.25 parts by weight) was weighed and sieved and added to the blended brigatinib mixture and again blended to distribute the magnesium stearate (typically 75 to 175 revolutions at 15 rpm).

The blended mixture was then compressed into tablet cores comprising 30 mg or 90 mg of brigatinib drug substance using a rotary tablet press. The press may be equipped with product specific tooling to provide identifying markers, e.g. embossed or debossed markers, on the surface of the compressed tablet cores.

For 30 mg tablets, the target individual and mean tablet core weight was 150 mg and the compression parameters were selected so as to provide a target hardness of 13 kg-force. For 90 mg tablets, the target individual and mean tablet core weight was 450 mg and the compression parameters were selected so as to provide a target hardness of 16 kg-force.

Tablet core samples were tested throughout production for average and individual tablet weight, hardness and physical defects.

Opadry II white film coating system (Colorcon®) was weighed and blended with water according to the manufacturer's specifications. The coating suspension was sprayed onto the tablet cores inside a perforated rotating coating pan to obtain a target weight gain of 4% based on 100 wt % of the tablet cores. Coating parameters were typically monitored throughout the coating process in order to ensure the target coating weight gain and the coating suspension was continually mixed throughout the coating process to prevent settling.

The finished tablets were then packaged using an appropriate packaging system, for example a blister pack or a bottle provided with a child-resistant closure.

The composition of brigatinib tablets prepared according to Example 1 is set out in Table 1 below.

TABLE 1

| | | | Target Quantity | | |
|---|---|---|---|---|---|
| | | Percent | (mg/tablet) | | |
| | Component | (w/w) | 30 mg | 90 mg | Function |
| Core | Brigatinib | 20.0 | 30.0 | 90.0 | Active |
| Tablet | | | | | Ingredient |
| | Lactose | 37.37 | 56.06 | 168.16 | Filler |
| | Monohydrate | | | | |
| | Microcrystalline | 37.38 | 56.07 | 168.17 | Filler |
| | cellulose | | | | |
| | Sodium starch | 3.00 | 4.50 | 13.50 | Disintegrant |
| | glycolate Type A | | | | |
| | Hydrophobic | 1.00 | 1.50 | 4.50 | Glidant |
| | colloidal silica | | | | |
| | Magnesium | 1.25 | 1.87 | 5.62 | Lubricant |
| | stearate | | | | |
| | Total Core | 100% | 150 mg | 450 mg | |
| Film Coat | Opadry II White | — | 6.0 | 18.0 | Coating |
| | Film Coating | | | | Agent |
| | Purified Water | | q.s. | q.s. | Solvent |

| | | |
|---|---|---|
| Total Tablet Weight (mg) | 156.0 | 468.0 |

### Example 2—Crystallization of Brigatinib

In order to obtain brigatinib drug substance having the particle size distribution and crystal form described in Example 1, the following crystallization process has been developed. Brigatinib (1 part by weight), 1-propanol (4.35 parts by weight) and water (0.77 parts by weight) were stirred at 55-65° C. until the brigatinib was dissolved. The solution was filtered through a 0.25 μm filtration cartridge and then concentrated to a volume of around 5.4 L per kg of brigatinib. 6.0 parts by weight 1-propanol was added and the solution was again concentrated to a volume of 5.4 L per kg of brigatinib. The addition of 1-propanol and concentration of the solution were repeated once or twice more until the water content of the solution was no more than 0.5% w/w.

The reaction mixture was then heated to approximately 90° C., followed by the addition of ethyl acetate (1.33 parts by weight). The mixture was cooled to approximately 80° C. and seed crystals of brigatinib Form A (0.005 parts by weight) was added. The crystallization mixture was cooled at a rate of around 15° C./hour to 0±5° C. for no longer than 30 hours. The solid product was then filtered and washed with cold ethyl acetate before drying under nitrogen and then at 55° C. until a constant weight was obtained. The crystalline brigatinib product was obtained at 98% yield (Form A, D₅₀=9.6 μm, D₁₀=2.7 μm, D₅₀=23.1 μm).

### Example 3—Excipient Stability Study

In order to test the stability of the brigatinib active drug substance with various excipients, a series of excipient compatibility studies was carried out. A selection of the excipients tested are provided in Table 2 below.

TABLE 2

| Ingredient | Function | Trade Name | Supplier |
|---|---|---|---|
| Brigatinib Drug | API* | N/A | ARIAD |
| Substance (as | | | Pharmaceuticals, |
| described in | | | Inc. |
| Example 2) | | | |
| Microcrystalline | Filler | Avicel ® | FMC BioPolymer |
| cellulose | | PH-102 | |
| Lactose | Filler | SuperTabe ® | DMV-Fonterra |
| monohydrate | | 14SD | |
| Dibasic calcium | Filler | Fujicalin ® | Fuji Chemical |
| phosphate | | | Industry Co. |
| Sodium starch | Disintegrant | Explotab ® | JRS Pharma, Inc. |
| glycolate | | | |
| Croscarmellose | Disintegrant | Ac-Di-Sol ® | FMC BioPolymer |
| sodium | | | |
| Hydrophobic | Glidant | Cab-O-Sil ® | Cabot Corporation |
| colloidal silica | | M-5P | |
| Magnesium | Lubricant | Hyqual ® | Mallinckrodt, Inc. |
| stearate | | | |
| Sodium lauryl | Wetting | N/A | Spectrum Chemical |
| sulfate | agent | | Manufacturing |
| | | | Corp. |

*API = Active Pharmaceutical Ingredient

All excipients used in the compatibility studies were pre-screened through a 20 mesh screen, except for magnesium stearate, which was pre-screened through a 40 mesh screen. Binary and ternary mixtures of brigatinib and excipients were prepared by combining the brigatinib drug substance with the excipient(s) in 20 mL scintillation vials and blending using an inversion mixer for 10 minutes. The compositions of 14 different formulations tested are set out in Table 2 below. The formulations were tested in both dry and wet conditions. The dry samples were used and sampled as prepared. The wet samples were triturated with distilled water in the amounts shown in Table 3.

TABLE 3

| | Formulation No. | | | | | | | | | | | | | |
|---|---|---|---|---|---|---|---|---|---|---|---|---|---|---|
| Ingredient | 1 | 2 | 3 | 4 | 5 | 6 | 7 | 8 | 9 | 10 | 11 | 12 | 13 | 14 |
| Brigatinib | 1.0^{a} | 1.0 | 1.0 | 0.9 | 0.9 | 0.9 | 0.9 | 0.9 | 1.0 | 1.0 | 1.0 | 1.0 | 1.0 | 1.0 |
| Avicel ® PH-102 | 9.0 | | | | | | | | 9.0 | | | | | |
| SuperTab ® 14SD | | 9.0 | | | | | | | 9.0 | 9.0 | 9.0 | 9.0 | 9.0 | 9.0 |
| Fujicalin ® | | | 9.0 | | | | | | | | | | | |
| Explotab ® | | | | 0.1 | | | | | | 0.1 | | | | |
| Ac-Di-Sol ® | | | | | 0.1 | | | | | | 0.1 | | | |
| Cab-O-Sil ® M-5P | | | | | | 0.1 | | | | | | 0.1 | | |
| SLS | | | | | | | 0.1 | | | | | | 0.1 | |
| Hyqual ® | | | | | | | | 0.1 | | | | | | 0.1 |
| Water^{b} | 2.0 | 2.0 | 2.0 | 0.2 | 0.2 | 0.2 | 0.2 | 0.2 | 1.8 | 2.2 | 2.2 | 2.2 | 2.2 | 2.2 |

^{a}Entries refer to the amount in grams of each component in each test sample.
^{b}Wet samples only

The vials containing the wet and dry blends were tested in stability chambers at 40° C. and 75% relative humidity (RH) and at 60° C. and ambient humidity for a period of eight weeks in each case. The samples were tested for visual appearance, brigatinib assay, and impurities of the brigatinib drug substance at the start of the test and at the end of the eight-week testing period. The results are provided in Tables 4 to 9.

TABLE 4
Visual Appearance, DRY samples

| | | 8 weeks | |
|---|---|---|---|
| | Initial | 40° C./75% RH | 60° C./ambient RH |
| 1 | Pale lavender | Light purple powder | Light purple powder |
| | powder, homogenous | | |
| 2 | Pale lavender & | Light purple powder | Light purple powder |
| | white powder, | | |
| | dispersed chunks of | | |
| | purple API | | |
| 3 | Pale lavender & | Light tan powder with | Tan powder with |
| | white powder, | brown specks | brown specks |
| | dispersed chunks of | | |
| | purple API | | |
| 4 | Purple powder, | Purple powder | Purple powder |
| | homogenous | | |
| 5 | Purple powder, | Purple powder with | Purple powder |
| | homogenous | lumps | |
| 6 | Purple powder with | Purple powder with | Purple powder with |
| | white powder | off-white specks | off-white specks |
| | dispersed | | |
| 7 | Purple powder | Purple powder with | Purple powder with |
| | | white specks | white specks |
| 8 | Pale lavender | Light purple powder | Purple powder |
| | powder | | |
| 9 | White powder with | Light purple powder | Light tan powder |
| | lavender powder | | |
| | dispersed, dispersed | | |
| | chunks of purple API | | |
| 10 | Pale lavender & | Light purple powder | Light purple powder |
| | white powder, | | |
| | dispersed chunks of | | |
| | purple API | | |
| 11 | Pale lavender & | Light purple powder | Light purple powder |
| | white powder, | | |
| | dispersed chunks | | |
| | of purple API | | |
| 12 | Pale lavender & | Light purple powder | Light purple powder |
| | white powder | with off-white specks | with off-white layers |
| 13 | Pale lavender & | Light purple powder | Light purple powder |
| | white powder, | | with off-white layers |
| | dispersed chunks | | |
| | of purple API | | |
| 14 | Pale lavender & | Off-white powder | Off-white powder |
| | white powder | | |

TABLE 5
Visual Appearance, WET samples

| | | 8 weeks | |
|---|---|---|---|
| | Initial | 40° C./75% RH | 60° C./ambient RH |
| 1 | Pale lavender & | Off-white powder | Light purple powder |
| | white powder, | | |
| | dispersed chunks of | | |
| | purple API | | |
| 2 | Pale lavender | Purple powder | Light purple powder |
| | powder, dispersed | | |
| | granules of purple | | |
| | API | | |
| 3 | Pale lavender | Yellow-tan powder | Mustard-tan powder |
| | powder, dispersed | | |
| | chunks of purple | | |
| | powder | | |
| 4 | Purple powder, | Purple powder | Purple powder |
| | homogenous | | |
| 5 | Purple powder, | Purple powder | Purple powder |
| | homogenous | | |
| 6 | Purple & white | Purple powder | Purple powder with |
| | powder | with white specks | white specks |
| 7 | Purple powder, | Purple powder | Purple powder with |
| | homogenous | with lumps | lumps |
| 8 | Purple powder, | Purple powder | Light purple powder |
| | homogenous | | |
| 9 | Pale lavender & | Off-white powder | Light tan powder |
| | white powder | | |
| 10 | Pale lavender & | Light tan powder | Tan powder with dark |
| | white powder, | with lumps | powder on top |
| | dispersed chunks of | | |
| | purple API | | |
| 11 | Pale lavender & | Tan powder with | Tan powder with lumps |
| | white powder, | lumps | |
| | dispersed chunks of | | |
| | purple API | | |
| 12 | Pale lavender & | Light purple | Light tan powder |
| | white powder, | powder | |
| | dispersed chunks of | | |
| | purple API | | |
| 13 | Pale lavender & | Tan paste | Tan paste with black top |
| | white powder, | | layer |
| | dispersed chunks of | | |
| | purple API | | |
| 14 | Pale lavender & | Light purple | Light tan powder |
| | white powder | powder | |

TABLE 6
Brigatinib Assay (% label claim), DRY samples

| | | 8 weeks | |
|---|---|---|---|
| | Initial | 40° C./75% RH | 60° C./ambient RH |
| 1 | 93.7 | 87.7 | 98.1 |
| 2 | 106.7 | 91.2 | 82.6 |
| 3 | 106.1 | 94.6 | 90.9 |
| 4 | 98.8 | 97.7 | 97.8 |
| 5 | 91.2 | 92.2 | 95.8 |
| 6 | 99.4 | 96.2 | 100.3 |
| 7 | 96.2 | 96.0 | 94.9 |
| 8 | 101.2 | 100.0 | 100.6 |
| 9 | 96.4 | 94.2 | 104.1 |
| 10 | 97.1 | 90.2 | 98.3 |
| 11 | 119.8 | 79.2 | 91.2 |
| 12 | 83.0 | 108.2 | 107.4 |
| 13 | 66.9 | 42.4 | 92.5 |
| 14 | 115.3 | 92.4 | 102.6 |

TABLE 7
Brigatinib Assay (% label claim), WET samples

| | | 8 weeks | |
|---|---|---|---|
| | Initial | 40° C./75% RH | 60° C./ambient RH |
| 1 | 102.1 | 89.6 | 122.4 |
| 2 | 102.2 | 99.7 | 94.9 |
| 3 | 93.2 | 66.1 | 30.3 |
| 4 | 102.2 | 108.3 | 116.7 |
| 5 | 100.7 | 106.7 | 111.5 |
| 6 | 105.8 | 114.2 | 118.5 |
| 7 | 101.2 | 103.1 | 114.4 |
| 8 | 107.2 | 110.4 | 119.8 |
| 9 | 108.0 | 95.4 | 86.6 |
| 10 | 101.3 | 96.0 | 85.2 |
| 11 | 87.4 | 83.5 | 65.0 |
| 12 | 97.7 | 93.2 | 79.8 |
| 13 | 61.0 | 50.5 | 38.7 |
| 14 | 94.1 | 97.0 | 94.4 |

TABLE 8
Brigatinib Impurities (%), DRY samples

| | | 8 weeks | |
|---|---|---|---|
| | Initial | 40° C./75% RH | 60° C./ambient RH |
| 1 | 0.11 | 0.12 | 0.35 |
| 2 | <LOQ | 0.12 | 0.50 |
| 3 | <LOQ | 0.93 | 3.0 |
| 4 | <LOQ | 0.12 | 0.35 |
| 5 | <LOQ | 0.12 | 0.61 |
| 6 | <LOQ | 0.12 | 0.58 |
| 7 | <LOQ | 0.12 | 0.37 |
| 8 | <LOQ | 0.12 | 0.34 |
| 9 | <LOQ | 0.12 | 0.33 |
| 10 | <LOQ | 0.34 | 0.45 |
| 11 | <LOQ | 0.26 | 0.36 |
| 12 | <LOQ | 0.43 | 0.59 |
| 13 | ND | 1.1 | 0.46 |
| 14 | <LOQ | 0.49 | 0.52 |

*<LOQ = below limit of quantification, ND = None detected

TABLE 9
Brigatinib Impurities (%), WET samples

| | | 8 weeks | |
|---|---|---|---|
| | Initial | 40° C./75% RH | 60° C./ambient RH |
| 1 | 0.21 | 1.8 | 5.1 |
| 2 | 0.22 | 0.66 | 5.0 |
| 3 | 0.29 | 31.0 | 62.6 |
| 4 | 0.10 | 0.12 | 0.19 |
| 5 | 0.12 | 1.4 | 1.3 |
| 6 | 0.21 | 0.35 | 0.61 |
| 7 | <LOQ | 0.63 | 0.99 |
| 8 | 0.22 | 0.12 | 0.18 |
| 9 | 0.30 | 1.9 | 7.5 |
| 10 | <LOQ | 1.7 | 8.3 |
| 11 | <LOQ | 14.8 | 30.7 |
| 12 | 0.12 | 2.96 | 11.8 |
| 13 | <LOQ | 8.3 | 2.7 |
| 14 | 0.33 | 0.12 | 3.2 |

The results of these experiments demonstrate that the stability of the brigatinib drug substance is significantly increased in the presence of microcrystalline cellulose and lactose monohydrate (Formulations 1 and 2) as compared to the conventional filler dibasic calcium phosphate (Formulation 3). Particularly in the case of wet samples, a significant deterioration in visual appearance, reduction in brigatinib assay and increase in brigatinib impurities is obtained in the presence of dibasic calcium phosphate.

A significant increase in the formation of brigatinib impurities is also observed with the use of the conventional croscarmellose sodium disintegrant (Formulation 5) as compared to the use of sodium starch glycolate (Formulation 4). The instability of brigatinib in the presence of croscarmellose sodium is amplified in the presence of lactose monohydrate filler as demonstrated by Formulations 10 and 11.

The inventors have further identified that the inclusion of the conventional wetting agent sodium lauryl sulfate has a deleterious effect on brigatinib stability—particularly in wet samples—as demonstrated by Formulation 13 (in comparison to, e.g., Formulation 2).

### Example 4—Co-Processing of Brigatinib with Colloidal Silicon Dioxide

The objective of the study was to evaluate the effects of a co-processing process (using brigatinib and colloidal silicon dioxide) on the manufacturing problems due to the stickiness of the pharmaceutical composition. Applying drug power coatings to an active pharmaceutical ingredient powders (of ibuprofen) using a comil has been reported in Mullarney et al., Powder Technology, 2011, 212:397-402. The effects of total number of comilling cycles and silica loading on the flow behaviour of a cohesive excipient powder (of microcrystalline cellulose) has been studied in Chattoraj et al., Journal of Pharmaceutical Sciences, 2011, 100(11):4943-4952.

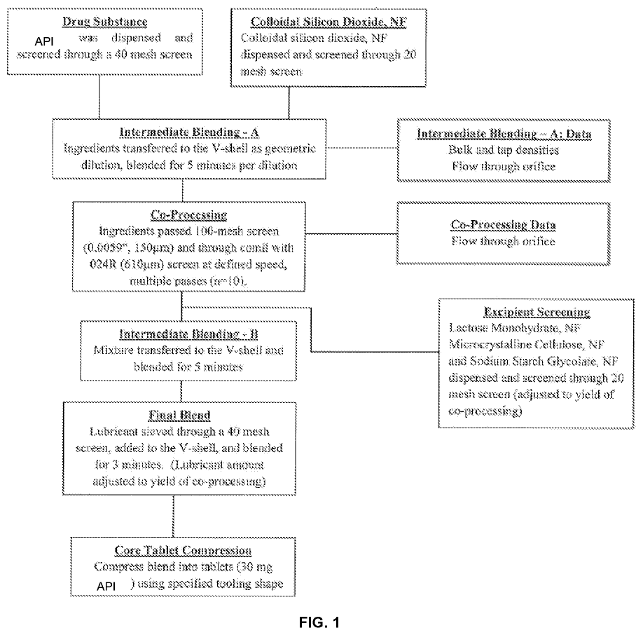

The study was carried out with two different lots of brigatinib (API), following the representative co-processing process depicted in FIG. 1. Aerosil R972® was selected as a hydrophobic grade of colloidal silicon dioxide for experimentation.

### Study No. 1

The formulation of study 1 (using a first lot of brigatinib) is shown in Table 10. The in-process data are provided in Table 11.

TABLE 10
Brigatinib Tablets, 30 mg Formulation

| Ingredients | % w/w | mg/dose |
|---|---|---|
| Brigatinib | 20.00 | 30.00 |
| Lactose, Monohydrate, NF | 37.38 | 56.07 |
| SuperTab 14SD ® | | |
| Microcrystalline Cellulose, NF | 37.38 | 56.07 |
| Avicel ® PH-102 | | |
| Sodium Starch Glycolate, NF | 3.00 | 4.50 |
| Explotab ® | | |
| Colloidal Silicon Dioxide, NF | 1.00 | 1.50 |
| Aerosil R972 ® | | |
| Magnesium Stearate, NF | 1.25 | 1.88 |
| Hyqual ®, vegetable source | | |
| Total | 100 | 150.0 |

TABLE 11
In-Process Data

| Process/Data Description | |
|---|---|
| Total Batch Size, g | 250 |
| Mill Device | Quadro ® Comil U3 |
| Screen Size | 024R (610 μm) |
| Impeller Speed, RPM | 2200 |

Pre-Blend (Brigatinib, colloidal silicon dioxide)-Initial

| | |
|---|---|
| Bulk Density, g/mL | 0.25 |
| Tap Density, g/mL | 0.41 |
| Hausner Ratio | 1.66 |
| Compressibility Index, % | 40 |
| Flow Through Orifice, mm (3X) | 26 |

Co-Processing (Comil Pass)

| | |
|---|---|
| Comil Pass No. 1, Flow Through Orifice, mm (3X) | 24 |
| Comil Pass No. 2, Flow Through Orifice, mm (3X) | 24 |
| Comil Pass No. 3, Flow Through Orifice, mm (3X) | 24 |
| Comil Pass No. 4, Flow Through Orifice, mm (3X) | 22 |
| Comil Pass No. 5, Flow Through Orifice, mm (3X) | 22 |
| Comil Pass No. 6, Flow Through Orifice, mm (3X) | 22 |
| Comil Pass No. 7, Flow Through Orifice, mm (3X) | Not Tested |
| Comil Pass No. 8, Flow Through Orifice, mm (3X) | Not Tested |
| Comil Pass No. 9, Flow Through Orifice, mm (3X) | Not Tested |
| Comil Pass No. 10, Flow Through Orifice, mm (3X) | 20 |
| Comil Pass No. 10, Bulk Density, g/mL | 0.26 |

Final Blend

| | |
|---|---|
| Flow Through Orifice, mm (3X) | 20 |

The flow through orifice data show improvement in flow characteristics, from 26 mm orifice at initial to 20 mm at the final tenth pass through the comil. Some amount of loss from the co-processing operation was experienced as seen in Table 12. The remaining ingredients in the formulation were adjusted by weight to compensate for the loss during the co-processing operation as shown in FIG. 1.

TABLE 12
Net Weight and Percent Loss of Pre-Blend in Co-Processing

| Description | Net Weight, g |
|---|---|
| Initial | 52.50 |
| Comil Pass No. 1 | 42.73 |
| Comil Pass No. 2 | 40.23 |
| Comil Pass No. 3 | 37.54 |
| Comil Pass No. 4 | 34.06 |
| Comil Pass No. 5 | 30.82 |
| Comil Pass No. 6 | 28.00 |
| Comil Pass No. 7 | 26.74 |
| Comil Pass No. 10 (final) | 24.70 |
| Loss | −27.80 |

### Study No. 2

The formulation of study 2 (using a second lot of brigatinib) is shown in Table 13. The in-process data are provided in Table 14.

TABLE 13
Brigatinib Tablets, 30 mg Formulation

| Ingredients | % w/w | mg/dose |
|---|---|---|
| Brigatinib | 20.00 | 30.00 |
| Lactose, Monohydrate, NF | 37.38 | 56.07 |
| SuperTab 14SD ® | | |
| Microcrystalline Cellulose, NF | 37.38 | 56.07 |
| Avicel ® PH-102 | | |
| Sodium Starch Glycolate, NF | 3.00 | 4.50 |
| Explotab ® | | |
| Colloidal Silicon Dioxide, NF | 1.00 | 1.50 |
| Aerosil R972 ® | | |
| Magnesium Stearate, NF | 1.25 | 1.88 |
| Hyqual ®, vegetable source | | |
| Total | 100 | 150.0 |

TABLE 14
In-Process Data

| Process/Data Description | |
|---|---|
| Total Batch Size, g | 250 |
| Mill Device | Quadro ® Comil U3 |
| Screen Size | 024R (610 μm) |
| Impeller Speed, RPM | 2200 |

Pre-Blend (Brigatinib, colloidal silicon dioxide)-Initial

| | |
|---|---|
| Bulk Density, g/mL | 0.30 |
| Tap Density, g/mL | 0.54 |
| Hausner Ratio | 1.83 |
| Compressibility Index, % | 45 |
| Flow Through Orifice, mm (3X) | 22 |

Co-Processing (Comil Pass)

| | |
|---|---|
| Comil Pass No. 1, Flow Through Orifice, mm (3X) | 22 |
| Comil Pass No. 2, Flow Through Orifice, mm (3X) | 22 |
| Comil Pass No. 3, Flow Through Orifice, mm (3X) | 22 |
| Comil Pass No. 4, Flow Through Orifice, mm (3X) | 16 |
| Comil Pass No. 5, Flow Through Orifice, mm (3X) | 16 |
| Comil Pass No. 6, Flow Through Orifice, mm (3X) | 16 |
| Comil Pass No. 7, Flow Through Orifice, mm (3X) | 18 |
| Comil Pass No. 8, Flow Through Orifice, mm (3X) | 16 |
| Comil Pass No. 9, Flow Through Orifice, mm (3X) | 18 |
| Comil Pass No. 10, Flow Through Orifice, mm (3X) | 14 |

Final Blend

| | |
|---|---|
| Bulk Density, g/mL | 0.49 |
| Tap Density, g/mL | 0.67 |
| Hausner Ratio | 1.37 |
| Compressibility Index, % | 27 |
| Flow Through Orifice, mm (3X) | 20 |

The flow through orifice data show improvement in flow characteristics, with some variability between 16 mm and 18 mm at comil pass four through nine. Approximately 21% of the brigatinib/colloidal silicon dioxide was lost from the co-processing operation as shown in Table 15.

TABLE 15
Net Weight and Percent Loss of Pre-Blend in Co-Processing

| Description | Net Weight, g |
|---|---|
| Initial | 52.50 |
| Comil Pass No. 1 | 49.43 |
| Comil Pass No. 2 | 47.88 |
| Comil Pass No. 3 | 48.54 |
| Comil Pass No. 4 | 47.66 |
| Comil Pass No. 5 | 46.24 |
| Comil Pass No. 6 | 45.49 |
| Comil Pass No. 7 | 43.78 |
| Comil Pass No. 8 | 42.16 |
| Comil Pass No. 9 | 41.70 |
| Comil Pass No. 10 (final) | 41.25 |
| Loss | −11.25 |

1. A pharmaceutical composition comprising:
(i) about 10 to about 40 wt % of 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib) or a pharmaceutically acceptable salt thereof;
(ii) about 20 to about 50 wt % of lactose monohydrate; and
(iii) about 15 to about 50 wt % of microcrystalline cellulose.

2. A pharmaceutical composition according to claim 1, further comprising about 0.2 to about 3 wt % of hydrophobic colloidal silica.

3. A pharmaceutical composition according to claim 1 or claim 2, further comprising about 0.5 to about 5 wt % of sodium starch glycolate Type A.

4. A pharmaceutical composition comprising:
(i) about 10 to about 40 wt % 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib) or a pharmaceutically acceptable salt thereof; and
(ii) about 0.2 to about 3 wt % hydrophobic colloidal silica.

5. A pharmaceutical composition according to claim 4, further comprising about 20 to about 50 wt % of lactose monohydrate and about 15 to about 50 wt % of microcrystalline cellulose.

6. A pharmaceutical composition according to claim 4 or claim 5, further comprising about 0.5 to about 5 wt % of sodium starch glycolate Type A.

7. A pharmaceutical composition comprising:
(i) about 10 to about 40 wt % 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib) or a pharmaceutically acceptable salt thereof; and
(ii) about 0.5 to about 5 wt % of sodium starch glycolate Type A.

8. A pharmaceutical composition according to claim 7, further comprising about 20 to about 50 wt % of lactose monohydrate and about 15 to about 50 wt % of microcrystalline cellulose.

9. A pharmaceutical composition according to claim 7 or claim 8, further comprising about 0.2 to about 3 wt % of hydrophobic colloidal silica.

10. A pharmaceutical composition according to any one of the preceding claims, comprising brigatinib or a pharmaceutically acceptable salt thereof in an amount of from about 12 to about 35 wt %, more preferably about 15 to about 30 wt % and most preferably about 18 to about 25 wt % based on the total weight of the pharmaceutical composition.

11. A pharmaceutical composition according to any one of the preceding claims, wherein the brigatinib is in the free base form.

12. A pharmaceutical composition according to any one of the preceding claims, comprising lactose monohydrate in an amount of from about 25 to about 45 wt %, more preferably about 30 to about 40 wt % and most preferably about 32 to about 38 wt % based on the total weight of the pharmaceutical composition.

13. A pharmaceutical composition according to any one of the preceding claims, comprising microcrystalline cellulose in an amount of from about 20 to about 45 wt %, more preferably about 25 to about 40 wt %, more preferably from about 30 to about 40 wt % and most preferably about 32 to about 38 wt % based on the total weight of the pharmaceutical composition.

14. A pharmaceutical composition according to any one of the preceding claims, comprising hydrophobic colloidal silica in an amount of from about 0.4 to about 2 wt %, more preferably from about 0.6 to about 1.5 wt %, and most preferably from about 0.8 to about 1.2 wt %.

15. A pharmaceutical composition according to any one of the preceding claims, comprising sodium starch glycolate Type A in an optimized amount of from about 1 to about 5 wt %, more preferably about 1.5 to about 4.5 wt %, and more preferably about 2 to about 4 wt %.

16. A pharmaceutical composition according to any one of the preceding claims, further comprising one or more lubricants.

17. A pharmaceutical composition according to claim 16 comprising magnesium stearate, optionally in an amount of from about 0.2 to about 3 wt %, from about 0.5 to about 2.5 wt %, from about 0.8 to about 2 wt % or from about 1 to about 1.8 wt %.

18. A pharmaceutical composition according to any one of the preceding claims, comprising or consisting of:
(i) about 10 to about 40 wt % of 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib);
(ii) about 20 to about 50 wt % of lactose monohydrate;
(iii) about 15 to about 50 wt % of microcrystalline cellulose;
(iv) about 0.5 to about 5 wt % of sodium starch glycolate Type A;
(v) about 0.2 to about 2 wt % of hydrophobic colloidal silica;
(vi) about 0.2 to about 3 wt % of magnesium stearate.

19. A pharmaceutical composition according to claim 18, comprising or consisting of:
(i) about 12 to about 35 wt % of 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib);
(ii) about 25 to about 45 wt % of lactose monohydrate;
(iii) about 20 to about 45 wt % of microcrystalline cellulose;
(iv) about 1 to about 5 wt % of sodium starch glycolate Type A;
(v) about 0.4 to about 1.8 wt % of hydrophobic colloidal silica;
(vi) about 0.5 to about 2.5 wt % of magnesium stearate.

20. A pharmaceutical composition according to claim 19, comprising or consisting of:
(i) about 15 to about 30 wt % of 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib);
(ii) about 30 to about 40 wt % of lactose monohydrate;
(iii) about 25 to about 40 wt % of microcrystalline cellulose;
(iv) about 1.5 to about 4.5 wt % of sodium starch glycolate Type A;
(v) about 0.6 to about 1.5 wt % of hydrophobic colloidal silica;
(vi) about 0.8 to about 2 wt % of magnesium stearate.

21. A pharmaceutical composition according to claim 20, comprising or consisting of:
(i) about 18 to about 25 wt % of 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib);
(ii) about 32 to about 38 wt % of lactose monohydrate;
(iii) about 30 to about 38 wt % of microcrystalline cellulose;
(iv) about 2 to about 4 wt % of sodium starch glycolate Type A;
(v) about 0.8 to about 1.2 wt % of hydrophobic colloidal silica;
(vi) about 1 to about 1.8 wt % of magnesium stearate.

22. A pharmaceutical composition according to claim 21, consisting of:
(i) about 20 wt % of 5-chloro-N4-[2-(dimethylphosphoryl)phenyl]-N2-{2-methoxy-4-[4-(4-methylpiperazin-1-yl)piperidin-1-yl]phenyl}pyrimidine-2,4-diamine (brigatinib);
(ii) about 36 to about 39 wt % of lactose monohydrate;
(iii) about 36 to about 39 wt % of microcrystalline cellulose;
(iv) about 3 wt % of sodium starch glycolate Type A;
(v) about 1 wt % of hydrophobic colloidal silica;
(vi) about 1.25 wt % of magnesium stearate.

23. A pharmaceutical composition according to any one of the preceding claims, wherein the brigatinib comprises at least about 50 wt %, at least about 60 wt %, at least about 70 wt %, at least about 80 wt %, at least about 90 wt %, at least about 95 wt %, at least about 98 wt % or at least about 99 wt % of brigatinib polymorphic Form A, based on the total amount of brigatinib.

24. A pharmaceutical composition according to any one of the preceding claims, wherein the brigatinib has a D₅₀ particle size in the range of from about 5 to about 25 μm, preferably from about 6 to about 25 μm, preferably from about 8 to about 22 μm, more preferably from about 10 to about 20 μm.

25. A pharmaceutical composition according to any one of the preceding claims, wherein the brigatinib has a D₁₀ particle size of at least about 0.5 μm, more preferably at least about 1 μm, more preferably at least about 1.5 μm, more preferably at least about 2 μm, more preferably at least about 2.5 μm, but no more than about 8 μm.

26. A pharmaceutical composition according to any one of the preceding claims, wherein the brigatinib has a D₉₀ particle size of no more than about 90 μm, more preferably no more than about 60 μm, more preferably no more than about 55 μm, more preferably no more than about 50 μm, more preferably no more than about 45 μm.

27. A pharmaceutical composition according to any one of claims 1 to 23, wherein the brigatinib has a D₅₀ particle size in the range of from 5 to 25 μm, preferably from 6 to 15 μm, more preferably from 8 to 10 μm.

28. A pharmaceutical composition according to any one of claims 1 to 23 and 27, wherein the brigatinib has a D₁₀ particle size of at least 1 μm, more preferably at least 1.5 μm, more preferably at least 1.8 μm, for example at least 2 μm, or at least 2.5 μm; and/or

29. A pharmaceutical composition according to any one of claims 1 to 23, 27 and 28, wherein the brigatinib has a D₉₀ particle size of no more than 40 μm, more preferably no more than 35 μm, more preferably no more than 30 μm, more preferably no more than 25 μm.

30. A pharmaceutical composition according to any one of the preceding claims, wherein the pharmaceutical composition is storage stable for at least 6 months at about 25° C. and about 60% relative humidity.

31. A pharmaceutical composition according to any one of the preceding claims, wherein the pharmaceutical composition is storage stable for at least 8 weeks at about 40° C. and about 75% relative humidity and/or for at least 8 weeks at about 60° C. and ambient humidity.

32. A pharmaceutical composition according to any one of the preceding claims, wherein the pharmaceutical composition is in a solid oral dosage form.

33. A pharmaceutical composition according to any one of the preceding claims in tablet form.

34. A pharmaceutical tablet comprising a tablet core comprising or consisting of a pharmaceutical composition as defined in any one of claims 1 to 33, and optionally a coating.

35. A pharmaceutical tablet according to claim 34, wherein the tablet core consists of a pharmaceutical composition as defined in any one of claims 18 to 22.

36. A pharmaceutical tablet according to claim 35, wherein the tablet core consists of a pharmaceutical composition as defined in claim 22.

37. A pharmaceutical tablet according to any one of claims 34 to 36, comprising a coating selected from polymeric coatings and sugar coatings.

38. A pharmaceutical tablet according to claim 37, wherein the coating is present in an amount of from about 0.5 to about 10 wt %, preferably about 1 to about 8 wt %, preferably about 2 to about 5 wt % based on about 100 wt % of the tablet core.

39. A pharmaceutical tablet according to claim 37 or claim 38, wherein the coating is present at a thickness of from about 20 to about 100 μm.

40. A pharmaceutical tablet according to any one of claims 37 to 39, wherein the coating polymer is selected from cellulose derivatives, such as cellulose ethers, acrylic polymers and copolymers, methacrylic polymers and copolymers polyethylene glycols, polyvinyl pyrrolidones, and polyvinyl alcohols.

41. A pharmaceutical tablet according to any one of claims 37 to 40, wherein the tablet coating is selected for immediate release of the brigatinib drug substance following ingestion of the tablets by a patient.

42. A pharmaceutical tablet according to any one of claims 34 to 41, comprising from about 5 to about 500 mg brigatinib, preferably from about 10 to about 250 mg brigatinib, and more preferably from about 20 to about 200 mg brigatinib.

43. A pharmaceutical tablet according to claim 42, comprising about 30 mg, about 90 mg or about 180 mg of brigatinib.

44. A method of preparing tablets comprising brigatinib, wherein the method comprises the steps of:
(i) blending brigatinib or a pharmaceutically acceptable salt thereof with one or more of lactose monohydrate, microcrystalline cellulose, hydrophobic colloidal silica, sodium starch glycolate, and magnesium stearate so as to obtain a pharmaceutical composition according to any of the first, second or third aspects of the invention; and
(ii) compressing the blended pharmaceutical composition to form a tablet core.

45. A method according to claim 44, wherein the brigatinib is in the free base form.

46. A method according to claim 44 or claim 45, wherein the method does not comprise at least one of a wet granulation step, a dry granulation step and a wet milling step.

47. A method according to any one of claims 44 to 46, wherein step (i) comprises the step of:
(ia) blending brigatinib and hydrophobic colloidal silica and passing the blended mixture of brigatinib and hydrophobic colloidal silica through a screening mill having a screen size in the range of from about 400 to about 800 μm.

48. A method according to claim 47, wherein (i) further comprises the step of:
(ib) blending the mixture from step (ia) with one or more of lactose monohydrate, microcrystalline cellulose, sodium starch glycolate, and magnesium stearate.

49. A method according to claim 47 or claim 48, wherein the mixture of brigatinib and hydrophobic colloidal silica in step (ia) is passed through the screening mill from 2 to 50 times, preferably from 5 to 20 times, for example 10 times.

50. A method according to any one of claims 44 to 49, wherein the brigatinib has a D₅₀ particle size in the range of from 5 to 25 μm, preferably from 6 to 15 μm, more preferably from 8 to 10 μm.

51. A method according to any one of claims 44 to 50, wherein the brigatinib has a D₁₀ particle size of at least 1 μm, more preferably at least 1.5 μm, more preferably at least 1.8 μm, for example at least 2 μm or at least 2.5 μm.

52. A method according to any one of claims 44 to 51, wherein the brigatinib has a D₉₀ particle size of no more than 40 μm, more preferably no more than 35 μm, more preferably no more than 30 μm, more preferably no more than 25 μm.

53. A method according to any one of claims 50 to 52, wherein the brigatinib is prepared by forming a solution of brigatinib in a mixture of 1-propanol and ethyl acetate at 70-90° C., adding seed crystals of brigatinib, and cooling the mixture at a rate of 10-20° C./hour to 0±5° C. for up to 30 hours, followed by separation of the brigatinib crystals from the crystallisation mother liquor.

54. A method according to any one of claims 44 to 53, wherein the pharmaceutical composition is compressed to form a tablet core in step (ii) using a rotary tablet press.

55. A method according to any one of claims 44 to 54, wherein the compression parameters in step (ii) are selected so as to obtain tablets having a hardness in the range of from about 10 to about 20 kg-force.

56. A method according to any one of claims 44 to 55, further comprising the step of:
(iii) providing the tablet core with a polymeric coating.

57. A method according to any one of claims 44 to 56, wherein the tablets are as defined in any one of claims 34 to 43.

58. A method of treating a disease or disorder responsive to the inhibition of ALK, the method comprising administering a pharmaceutical composition as defined above to a patient in need of such treatment.

59. A pharmaceutical composition as defined above for use in a method of treating a disease or disorder responsive to the inhibition of ALK, the method comprising administering a pharmaceutical composition as defined above to a patient in need of such treatment.

60. A method according to claim 58 or a pharmaceutical composition for use according to claim 59, wherein the pharmaceutical composition is in the form of a tablet according to any one of claims 34 to 43.

61. A method according to claim 58 or 60 or a pharmaceutical composition for use according to claim 59 or 60, wherein the disease or disorder responsive to the inhibition of ALK is an ALK+ driven cancer, such as non-small cell lung cancer, in particular ALK-positive non-small cell lung cancer.

62. A method according to any one of claims 58, 60 and 61 or a pharmaceutical composition for use according any one of claims 59 to 61, wherein the pharmaceutical composition is administered as a single dose of about 180 mg brigatinib per day or as a single dose of about 90 mg brigatinib per day for seven days, followed by a single dose of about 180 mg brigatinib per day.
